(19) United States
(12) Patent Application Publication
(10) Pub. No.: US 2025/0104816 A1
(43) Pub. Date: Mar. 27, 2025

(54) SYSTEM AND METHOD FOR CONFORMATION SAMPLING

(71) Applicant: QuanMol Tech, Inc., Hillsborough, CA (US)

(72) Inventors: Jie Li, San Carlos, CA (US); Minzhen Yi, Hillsborough, CA (US); Bo Li, San Carlos, CA (US); Jieyu Lu, San Carlos, CA (US); Xudong Lv, Pasadena, CA (US); Xingyu Shen, San Carlos, CA (US)

(21) Appl. No.: 18/472,171

(22) Filed: Sep. 21, 2023

(51) Int. Cl. G16C 20/50; G16B 15/00; G16C 20/40; G16C 20/64; G16C 20/70

(52) U.S. Cl. CPC G16C 20/50; G16B 15/00; G16C 20/40; G16C 20/64; G16C 20/70

(57) ABSTRACT

This disclosure presents a method and system aimed at improving the efficiency of conformation sampling for drug discovery or molecular design. An example method employs an iterative process with energy evaluations and Monte Carlo sampling to create a conformation pool. Initially, it calculates the energy of a ligand's initial conformation and detects rotatable bonds. The Monte Carlo algorithm randomly samples and rotates these bonds to generate new conformations, whose energies are assessed. Favorable, lower-energy conformations are directly stored, while higher-energy ones may be stored based on calculated probabilities inversely related to energy differences. The process continues iteratively until a specified exit condition is met, signifying convergence. Notably, this method extends beyond conformational analysis to offer binding guidance, facilitating the design of ligands with enhanced affinity and specificity.

### FIELD

The disclosure relates generally to molecular modeling and computational chemistry, particularly focusing on the conformational analysis of ligands in the context of protein-ligand binding interactions for drug discovery and other molecular modeling applications.

### BACKGROUND

The design and development of novel pharmaceutical compounds often hinge upon the precise understanding of how a ligand interacts with its target protein. This knowledge is crucial for optimizing drug candidates, improving binding affinities, and minimizing potential adverse effects.

Conformational analysis, which involves studying the different spatial arrangements or conformations that a ligand can adopt, plays a pivotal role in this endeavor. Specifically, identifying key rotatable bonds within a ligand molecule is essential. These rotatable bonds are responsible for the ligand's ability to flex and adapt its shape to the binding site of a protein.

Moreover, understanding the energy difference between a ligand's dominant conformation in an aqueous solution and its conformation when bound to a polymer is paramount. If a substantial energy difference exists between these two states, it can hinder ideal binding geometry, making the binding process more challenging. The presence of energy barriers due to difficult-to-rotate bonds can limit the effectiveness of potential drug candidates.

In existing methods, conformational analysis and the identification of key rotatable bonds in ligands have been time-consuming and computationally intensive processes. Accurate determination of energy differences between ligand conformations has often required resource-intensive computational techniques.

To address these challenges and advance the field of molecular modeling, the present disclosure describes a novel conformation analysis process that involves using Monte Carlo sampling algorithm to generate a reasonably representative pool of conformations with a reduced number of sampling rounds. This streamlined conformation analysis process allows researchers to make informed decisions in fields such as drug discovery, molecular dynamics simulations, and protein-ligand binding studies.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In some aspects, the techniques described herein relate to a computer-implemented method, including: executing an iterative process using a first molecular conformation of a ligand molecule to obtain a conformation pool, wherein the iterative process includes: determining a first conformation energy of the first molecular conformation; detecting one or more rotatable bonds in the first molecular conformation based on geometry information of the first molecular conformation; randomly sampling, using a Monte Carlo sampling algorithm, a rotatable bond from the one or more detected rotatable bonds and rotating the rotatable bond to obtain a second molecular conformation; determining a second conformation energy of the second molecular conformation; in response to the second conformation energy being less than the first conformation energy, storing the second conformation in a conformation pool; in response to the second conformation energy being greater than the first conformation energy, determining a probability to store the second conformation in the conformation pool, wherein the probability is exponentially inversely related to a difference between the first conformation energy and the second conformation energy; replacing the first molecular conformation with the second molecular conformation if the second conformation is stored in the conformation pool; and continuing the iterative process until an exit condition is met; and generating binding guidance for the first molecular conformation based on conformations stored in the conformation pool.

In some aspects, the generating the binding guidance includes: determining one or more key rotatable bonds of the first molecular conformation based on bond rotation angle statistics of conformations in the conformation pool, wherein the one or more key rotatable bonds correspond to rotation angles with the least number of appearances in the conformation pool; and visualizing, on a graphic user interface (GUI), the first molecular conformation by highlighting the one or more key rotatable bonds and displaying corresponding statistics in a histogram.

In some aspects, the highlighting the one or more key rotatable bonds includes: displaying the one or more key rotatable bonds using a color different from a color of other rotatable bonds in the first molecular conformation.

In some aspects, the generating the binding guidance includes: generating conformation entropy based on conformations in the conformation pool.

In some aspects, the computer-implemented method may further include: converting cartesian coordinates of atoms in the first molecular conformation into internal coordinates before detecting the one or more rotatable bonds in the first molecular conformation.

In some aspects, the determining the second conformation energy of the second molecular conformation includes: inputting the second molecular conformation into a pre-trained deep neural network to evaluate conformation energies of given molecular conformations, wherein the pre-trained deep neural network is trained with labeled training data including: a plurality of molecular structures represented by atomic coordinates, and corresponding quantum mechanical calculated energies as labels.

In some aspects, the pre-trained deep neural network includes ANI-2 (ANAKIN-ME model version 2).

In some aspects, the detecting one or more rotatable bonds in the first molecular conformation includes: inputting the geometry information of the first molecular conformation into a software application to check bond types and determine whether a bond is in a ring or has other geometrical constrains.

In some aspects, the software application includes AutoDockTools or RDKit.

In some aspects, the rotating the rotatable bond to obtain the second molecular conformation includes: rotating the rotatable bond by a degree (e.g., 60 degree with a perturbation degree) using molecular modeling software.

In some aspects, the computer-implemented method may further include: identifying key rotatable bonds that are most difficult to rotate based on conformations in the conformation pool; and creating energy barriers for the key rotatable bonds before binding the ligand molecule to a protein molecule or a polymer.

In some aspects, the generating binding guidance for the first molecular conformation includes: selecting one conformation from the conformation pool based on (1) a conformation energy of the selected conformation and (2) an amount of energy needed to transition the first molecular conformation to the selected conformation.

In some aspects, the continuing the iterative process until the exit condition is met includes: tracking an absolute difference between the first conformation energy and the second conformation energy for a plurality of steps in the iterative process; and exiting the iterative process when variations of the tracked absolute differences remain below a threshold for the plurality of steps.

In some aspects, the techniques described herein relate to a system including: one or more processors configured to: execute an iterative process for a first molecular conformation to obtain a conformation pool, wherein the iterative process includes: determine a first conformation energy of the first molecular conformation detect one or more rotatable bonds in the first molecular conformation based on geometry information of the first molecular conformation randomly sample, using a Monte Carlo sampling algorithm, a rotatable bond from the one or more detected rotatable bonds and rotating the rotatable bond to obtain a second molecular conformation determine a second conformation energy of the second molecular conformation in response to the second conformation energy be less than the first conformation energy, storing the second conformation in a conformation pool in response to the second conformation energy be greater than the first conformation energy, determining a probability to store the second conformation in the conformation pool, wherein the probability is exponentially inversely related to a difference between the first conformation energy and the second conformation energy; replace the first molecular conformation with the second molecular conformation if the second conformation is stored in the conformation pool; and continue the iterative process until an exit condition is met; and generate binding guidance for the first molecular conformation based on conformations stored in the conformation pool.

In some aspects, the techniques described herein relate to a non-transitory computer-readable medium storing a set of instructions, the set of instructions including: one or more instructions that, when executed by one or more processors of a device, cause the device to: execute an iterative process for a first molecular conformation to obtain a conformation pool, wherein the iterative process includes: determine a first conformation energy of the first molecular conformation detect one or more rotatable bonds in the first molecular conformation based on geometry information of the first molecular conformation randomly sample, using a Monte Carlo sampling algorithm, a rotatable bond from the one or more detected rotatable bonds and rotating the rotatable bond to obtain a second molecular conformation determine a second conformation energy of the second molecular conformation in response to the second conformation energy be less than the first conformation energy, storing the second conformation in a conformation pool in response to the second conformation energy be greater than the first conformation energy, determining a probability to store the second conformation in the conformation pool, wherein the probability is exponentially inversely related to a difference between the first conformation energy and the second conformation energy; replace the first molecular conformation with the second molecular conformation if the second conformation is stored in the conformation pool; and continue the iterative process until an exit condition is met; and generate binding guidance for the first molecular conformation based on conformations stored in the conformation pool.

### BRIEF DESCRIPTION OF THE DRAWINGS

Preferred and non-limiting embodiments of the invention may be more readily understood by referring to the accompanying drawings in which:

FIG. 1 illustrates an example ligand with rotatable bonds.

FIG. 2 illustrates an example system integrating ligand conformation sampling and ligand-receptor binding optimization, in accordance with some embodiments.

FIG. 3 illustrates an example method integrating ligand conformation sampling and ligand-receptor binding optimization, in accordance with some embodiments.

FIG. 4 illustrates a block diagram of an example computer system in which any of the embodiments described herein may be implemented.

### DETAILED DESCRIPTION

Specific, non-limiting embodiments of the present invention will now be described with reference to the drawings. It should be understood that particular features and aspects of any embodiment disclosed herein may be used and/or combined with particular features and aspects of any other embodiment disclosed herein. It should also be understood that such embodiments are by way of example and are merely illustrative of a small number of embodiments within the scope of the present invention. Various changes and modifications obvious to one skilled in the art to which the present invention pertains are deemed to be within the spirit, scope, and contemplation of the present invention as further defined in the appended claims.

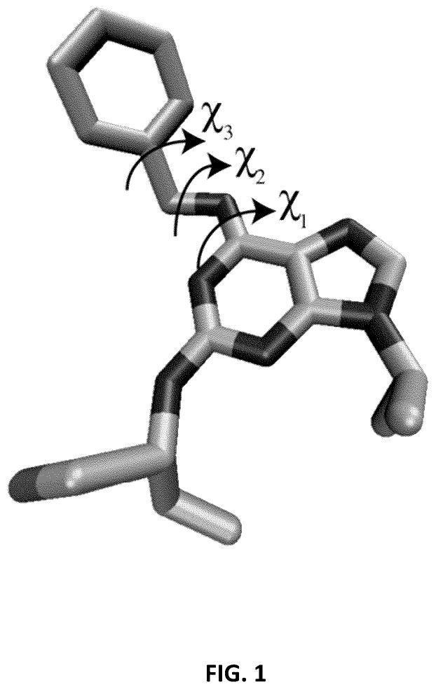

When a molecule, such as a ligand in FIG. 1, has rotatable bonds, it means that it contains specific chemical bonds that can rotate freely around their axis. As shown in FIG. 1, the rotatable bonds in the ligand are illustrated with arrows. These rotatable bonds are typically single bonds (sigma bonds) connecting two atoms, and they allow the molecule to adopt different conformations or spatial arrangements. In a given ligand, the presence of rotatable bonds therein is essential in molecular modeling, especially in the context of molecular docking and conformational analysis. These bonds give flexibility to the ligand, allowing it to adjust its shape to fit into the binding site of a target protein or receptor. This flexibility is crucial for understanding how the ligand interacts with the target and for predicting the most energetically favorable binding mode.

Scientists often analyze the rotatable bonds in a ligand to explore various conformations and determine which conformations are most likely to form strong interactions with the receptor. The selected conformations maximize binding affinity and minimizes steric hindrance or unfavorable interactions within the receptor's binding site.

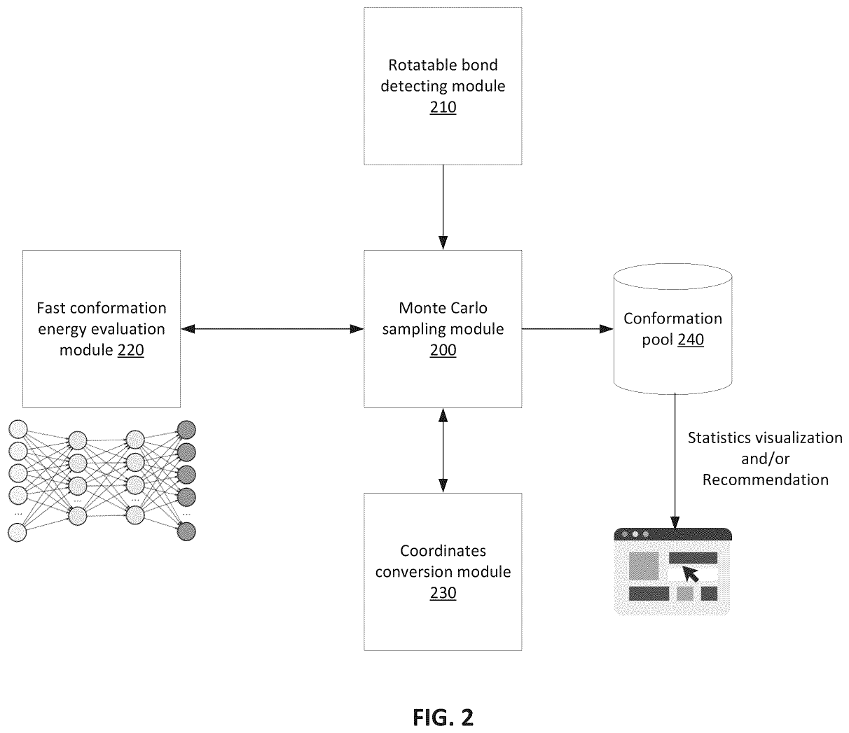

FIG. 2 illustrates an example system integrating ligand conformation sampling and ligand-receptor binding optimization, in accordance with some embodiments. The multiple modules in FIG. 2 are for illustrative purposes. Depending on the implementation, the system may include more, fewer, or alternative modules. The modules can be implemented on a server device or a cloud service, with a user-interface (e.g., a graphic user-interface (GUI) or a command-line (CLI) interface) for interacting with researchers. For example, the visualization and statistics of the key rotatable bonds in a ligand may be displayed to a researcher. The user-interfaces can take the form of either a desktop application or a web-based application. For example, the user-interface might involve a desktop application installed directly on a user's computer or a web-based application accessed through a web browser. The service device or the cloud service is responsible for sending computed data to the desktop application or the web-based application via internet connections.

In some embodiments, the example system includes a rotatable bond detecting module 210, a fast conformation energy evaluation module 220, a coordinates conversion module 230, a Monte Carlo sampling module 200, and a storage medium for storing a conformation pool 240 (i.e., a pool of selected conformations). In some embodiments, statistics and visualizations may be generated based on the conformations in the conformation pool 240 and displayed on a GUI.

The rotatable bond detecting module 210 may be configured to detect rotatable bonds in a given molecular conformation. The rotatable bond detecting module 210 may check bond types and determine whether a bond is in a ring or has other geometrical constrains in order to determine whether the bond is rotatable. Molecular modeling software packages, such as AutoDockTools, Open Babel, RDKit, and Cheminformatics Toolkit, provide libraries and functions to detect rotatable bonds automatically. Additionally, programming languages like Python can be used to create custom scripts for this purpose, making it easier to integrate into your computational workflow.

In some embodiments, the rotatable bond detecting module 210 may be implemented as a machine learning (ML) model. The ML model may be trained based on a dataset of molecular conformations with labeled rotatable bonds (e.g., labels may be generated manually). The ML model may accept geometry information of a given molecular conformation as input (e.g., 3D representation of the conformation, atom types, bond types, distances, angles, and other structural properties), and be trained to output predicted labels for the bonds in the conformation. The differences between the predicted labels and the ground-truth labels may be used to determine a loss, which may be used to tune the parameters of the ML model to minimize the future loss. The ML model may include one or more feature extracting layers to first identify the single bonds in the conformation (connecting two atoms), check bond types and connectivity to exclude the bonds connecting two same atoms or is part of a ring structure, identify bonds connected to terminal atoms, etc.

In some embodiments, the output of the rotatable bond detecting module 210 may include a plurality of detected rotatable bonds in the given ligand conformation. These detected rotatable bonds can then serve as input for the Monte Carlo sampling module 200. The Monte Carlo sampling module 200 collaborates closely with the fast conformational energy evaluation module 220, as it relies on conformational energy estimates to guide the sampling process.

The Monte Carlo sampling module 200 is designed to iteratively sample and select “stable” conformations over a limited number of iterations, thereby acquiring a reasonably representative set of conformations for the specified ligand. This sampling approach offers greater efficiency compared to random sampling, as it minimizes the selection of “unstable” conformations, consequently conserving computational resources by avoiding unnecessary computations for all conformations derived from these “unstable” ones. Here, the stable conformations refer to the ones with low conformation energies, e.g., lower than a threshold. The unstable conformations refer to the ones with high conformation energies, e.g., greater than the threshold.

For example, the Monte Carlo sampling module 200 may first randomly select a rotatable bond from the output of the rotatable bond detecting module 210, and rotate the bond for a specified degree. The specified degree may be 60 degree for balanced torsional angle resolution. In many molecular modeling software packages and force fields, the torsional angle (dihedral angle) resolution is set to 60 degrees. This means that the software calculates energy and geometry at intervals of 60 degrees for torsion angles. Therefore, performing 60-degree rotations aligns with this resolution and facilitates energy calculations. In some embodiments, a small perturbation degree may be introduced (e.g., 5 degree) to the rotation of the bond for better exploration of local minima (e.g., small perturbations can facilitate the exploration of local minima within the energy landscape, which might be missed with larger perturbations like 60 degree). In some embodiments, the rotation degree may be randomly selected for better coverage of the sampling space.

Following the bond rotation, the resulting conformation is then fed into the fast conformational energy evaluation module 220 for conformational energy assessment. Subsequently, the energy of the newly formed conformation is compared to the energy of the original ligand conformation (prior to rotation). The original conformational energy can also be generated by the fast conformational energy evaluation module 220, using the original ligand conformation as input.

If the newly generated conformation exhibits a lower energy level compared to the original conformation, it is deemed more stable and is consequently added to the conformation pool 240. Conversely, if the new conformation possesses a higher energy than the original one, the Monte Carlo sampling module 200 calculates the probability of accepting this new conformation into the conformation pool 240. This probability is inversely proportional to the difference between the energy of the new conformation and that of the original conformation. In simpler terms, the greater the energy of the new conformation, the less likely it is to be retained within the conformation pool 240. If the new conformation is not accepted, the Monte Carlo sampling module may repeat the above process using a different rotatable bond.

If the new conformation is accepted and stored, it assumes the role of the starting point for the subsequent sampling iteration. Importantly, as the rotation of one bond in the conformation does not lead to significant changes in the remaining rotatable bonds (for example, from the original conformation to the new one), the information provided by the rotatable bond detecting module 210 remains applicable. In the subsequent sampling iteration, the Monte Carlo sampling module 200 may proceed to select another rotatable bond within the conformation, rotate it to generate a third conformation, and then carry out energy assessment and comparison anew. This iterative process continues as additional conformations are generated and evaluated for inclusion in the conformation pool.

In some embodiments, the iterative process stops when an exit condition is met. For instance, within the Monte Carlo sampling module 220, one way to determine this condition is by monitoring the sequence of absolute energy differences throughout the sampling iterations. If, in the last predetermined number of steps, the variations in the sequence of absolute values remain below a certain threshold, this suggests that the sampling process has likely converged and can be concluded. In some embodiments, the exit condition may include a pre-determined maximum number of iterations or the maximum number of conformations to be stored in the conformation pool. In these cases, even if the energy variation has converged, the iterative process continues to sample close to equilibrium geometries until the maximum number is reached.

In some embodiments, the fast conformational energy evaluation module 220 may include a deep neural network that is trained to predict the energy of an input conformation. The fast conformational energy evaluation module 220 may use existing tools such as ANI-2 (ANAKIN-ME model version 2), or train a more-customized deep neural network to accommodate the input format of the conformations, the specific types of ligands, etc.

For instance, the training data for training the fast conformational energy evaluation module 220 may include a diverse dataset of molecular structures, each represented by its atomic coordinates (Cartesian coordinates) and the corresponding quantum mechanical (QM) calculated energies. The QM calculated energies are used as ground truth for tuning the parameters of the deep neural work during training. The training data may first go through a preprocessing step for feature extraction (e.g., converting the Cartesian coordinates into input vectors including atom positions, bond lengths, bond angles, and dihedral angles (torsion angles)) and/or normalization (normalizing the input features and the energy values to have zero mean and unit variance for stabilizing the network). During the training process, the training data is first propagated through the layers of the network to obtain a loss (e.g., an error computed based on the difference between the predicted energy and the ground truth energy), and then backpropagation and gradient descent-based optimization techniques may be used to minimize the loss by tuning the weights in layers. The training may iterate for a plurality of rounds until the loss converges.

In some embodiments, the coordinates conversion module 230 may be configured to convert Cartesian coordinates to Internal coordinates thereby facilitating the computation in the Monte Carlo sampling module 200 and/or the fast conformational energy evaluation module 220. For instance, an input molecular conformation may be initially represented using Cartesian coordinates, in which the positions of atoms of the conformation are represented in a three-dimensional space using x, y, and z coordinates. Each atom is specified by its x, y, and z coordinates relative to a fixed reference point. The coordinates conversion module 230 may convert the input Cartesian coordinates to Internal coordinates, which represent the positions of atoms in terms of bond lengths, bond angles, and dihedral angles (torsion angles), etc. Instead of specifying the absolute positions of atoms, internal coordinates describe how atoms are connected within the molecule.

Conversely, the coordinates conversion module 230 may also revert internal coordinates back to Cartesian coordinates for specific purposes, such as storing a conformation in the conformation pool 240. In this case, the module transforms the internal coordinates into Cartesian coordinates to record the conformation's absolute atom positions in three-dimensional space.

Once the conformation pool 240 is established, downstream applications may consume the data in the conformation pool 240 to perform different analysis and guide the binding between ligand and receptor.

### Identifying Key Rotatable Bonds

Here, the key rotatable bonds refer to the difficult-to-rotate bonds, which have a low chance to be near the required angle for the conformation in the protein-ligand complex. These key rotatable bonds may be determined by bond rotation angle statistics of the conformations in the pool. For instance, these key rotatable bonds may be determined by calculating the proportion of each rotatable bond's starting angle plus or minus a small value (i.e. 5 degrees) in the distribution of all rotation angle values of this rotatable bond in the collected conformer pool, and selecting one or more bonds with the lowest proportion to be effective (close to the starting bond angle).

Once the difficult-to-rotate bonds are identified, medicinal chemists and drug designers can focus their efforts on optimizing that specific bonds. By making targeted modifications to the ligand in the vicinity of these bonds, they can alter their flexibility or steric, potentially improving the ligand's ability to adopt a conformation that is favorable for binding to the target.

For example, the difficult-to-rotate bonds provide insights into the energetic landscape of the ligand. Lowering the energy barriers associated with difficult-to-rotate bonds can facilitate the ligand's transition from its free form to the bound state. This reduction in energy barriers can be achieved through structural modifications, such as removing nearby groups that create steric hinderance to prevent the bond from rotating to its required position.

Conversely, a different scenario may involve a section of the ligand displaying excessive flexibility (e.g., the easiest-to-rotate bonds). Here, researchers may introduce steric hindrance by adding specific groups to “lock” the ligand conformation into an optimal state for binding with the protein. Therefore, this functionality can be utilized in various ways to address diverse molecular challenges, such as introducing macrocycles so that flexible side chains will be “locked” in place that facilitates interaction with the protein.

### Computing Entropy of Bond Rotation Angles

This calculation is pivotal in determining whether a particular bond can freely rotate when the ligand is not bound to the protein. Low entropy values indicate that ligand conformations predominantly adopt the same rotation angle for that bond, potentially signaling steric hindrance that users should address. Conversely, high entropy values suggest that the bond can rotate freely. In such cases, a researcher may introduce specific groups at strategic positions to curtail unrestricted rotation, encouraging the adoption of a torsion angle that optimally facilitates binding.

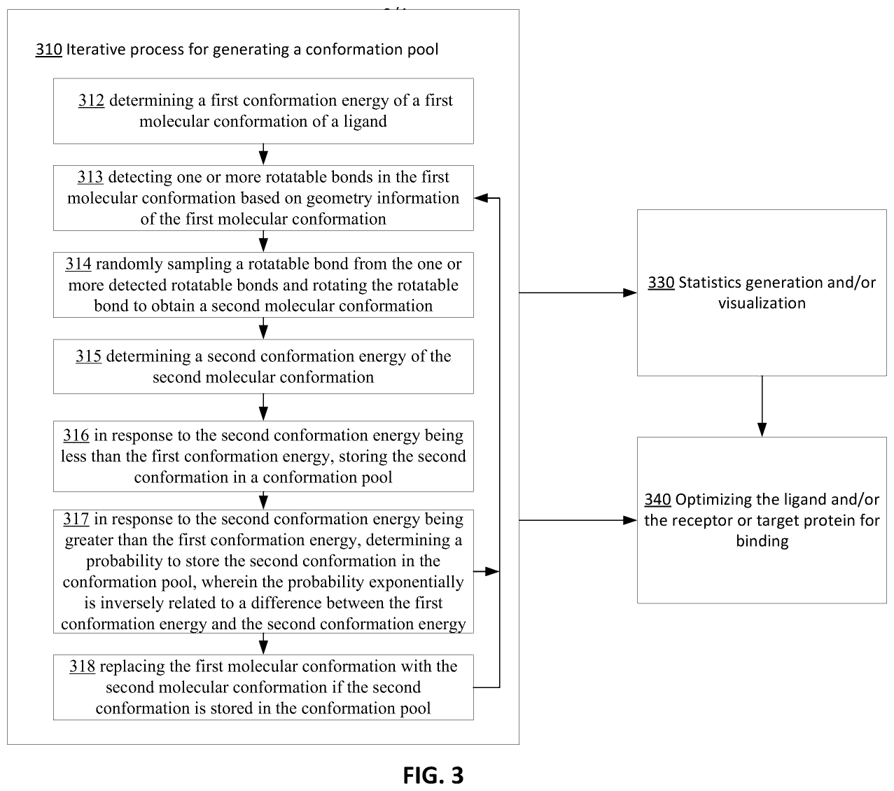

FIG. 3 illustrates an example method integrating ligand conformation sampling and ligand-receptor binding optimization, in accordance with some embodiments. In some implementations, one or more process blocks of FIG. 3 may be performed by a device.

The example method in FIG. 3 includes three core steps: an iterative process for generating a conformation pool (step 310), generating conformation statistics and visualizing key rotatable bond (step 330), and optimizing the ligand and/or the receptor or target protein for binding (step 340). The last two steps may be considered as downstream consumers of the output of the first step.

In some embodiments, the iterative process in step 310 may execute a pipeline for a plurality of rounds for collecting a pool of “stable” (low-energy) conformations. Given that the generation of conformations for a given ligand is computationally demanding, even when employing random sampling methods, the primary aim of the pipeline is to employ an innovative sampling approach that minimizes the number of sampling steps required while still producing a conformation pool that is reasonably representative.

In some embodiments, an initial input to the pipeline is a conformation of a ligand in aqueous solution. The pipeline may start with determining (step 312) a first conformation energy of the received conformation of the ligand. The first conformation energy may be generated by a trained deep neural network, such as ANI-2 or the customized model described in FIG. 2 (220). Then the first conformation may be input into a software application (e.g., AutoDockTools or RDKit) for detecting one or more rotatable bonds therein based on geometry information of the conformation (step 313). The software application may check bond types and determine whether a bond is in a ring or has other geometrical constrains based on the geometry information of the conformation. The geometry information of the conformation may use Cartesian coordinates, which may be converted into Internal coordinates before detecting the one or more rotatable bonds.

Next, one of the detected rotatable bonds may be selected (step 314) for rotation. The rotation may include rotating the bond for a pre-determined degree to generate a second conformation. In some embodiments, the pre-determined degree include a small random perturbation degree (smaller than 5 degree).

The newly generated second conformation may then input into the trained deep neural network for determining the second conformation energy (step 315). The first conformation energy and the second conformation energy may be compared. In response to the second conformation energy being less than the first conformation energy, the second conformation may be stored in a conformation pool (step 316). In response to the second conformation energy being greater than the first conformation energy, a probability may be computed based on the difference between the two energies to determine whether to store the second conformation in the conformation pool, wherein the probability is inversely related to the difference between the two energies (step 317) with an exponential decay.

If the newly generated second conformation has a higher energy (unstable) and not accepted/stored in the conformation pool, it is deemed abandoned and the iterative process 310 continues with the first conformation. If the second conformation has a lower energy (stable) or a marginally higher energy but stored in the conformation pool, the second conformation is adopted and used as the new starting conformation for the next round of iteration (step 318).

The iterative process 310 may continue until the energy variation among the conformations from a certain number of iteration steps is below a threshold. That is, when rotating bonds fail to increase or decrease the conformation energy by a threshold value, the iterative process 310 may considered converged. In some cases, the iterative process 310 may continue after the convergence until a desired number of iterations or conformations stored in the pool. The above-described sampling process borrows the idea from Monte Carlo sampling algorithm. It is different from traditional pure random sampling methods in which a rotatable bond is randomly selected and rotated based on the same initial conformation. In contrast, the Monte Carlo sampling algorithm based approach evolves the starting conformation each time a new conformation is accepted and stored in the pool.

With the conformation pool being generated, a plurality of statistics of the bonds in the conformation may be generated and/or visualized (step 330). For instance, one or more key rotatable bonds of the ligand conformation may be identified based on statistics of bond rotation angles in the conformer pool. For instance, they key rotatable bonds may correspond to rotation angles with the least number of appearances in the conformation pool. In other words, the key rotatable bonds may be the most-difficult-to-rotate bonds or the most-flexible rotatable bonds that they cannot stay at a certain angle that is required. The one or more key rotatable bonds may be visualized in a GUI for researchers to quickly capture the key rotatable bonds. For instance, the key rotatable bonds and the non-key rotatable bonds may use different colors for visualization. In addition, the angle distribution of that specific bond in the ensemble may also be displayed to help the user understand the energy landscape

The knowledge of the key rotatable bonds allows for more informed drug design. Researchers can focus on optimizing these specific bonds to enhance the binding affinity and specificity of a ligand to its target receptor. This can lead to the development of more effective pharmaceutical compounds. Also, when performing molecular simulations or conformational searches, concentrating computational resources on the optimization of key rotatable bonds can lead to significant time and cost savings. This focused approach reduces the need for exhaustive sampling of all bond rotations. Understanding which bonds are crucial for binding and which are more flexible helps in predicting ligand-receptor binding modes more accurately. It enables the identification of binding poses that are more likely to occur in vivo, improving the accuracy of binding affinity predictions. More importantly, researchers can tailor or optimize ligand designs to favor specific conformations that maximize interactions with the receptor by considering the key rotatable bonds (step 340). This optimization can result in ligands that exhibit higher binding affinities and improved therapeutic properties.

For instance, the ligand optimization (step 340) may include introducing constraint or rigidification (e.g., replacing a key bond, usually a single bond, with a double bond or introduce a ring structure to restrict movement in that region, or creating energy barriers for the key rotatable bonds before binding the ligand molecule to a protein molecule or a polymer), functional group modification (e.g., changing the size, polarity, or charge of the functional groups attached to key rotatable bonds to better complement the receptor's binding site), substitution or analog design (e.g., substituting atoms or functional groups on key rotatable bonds with chemically similar but more favorable groups), steric hindrance (e.g., if a key rotatable bond interferes with binding due to steric hindrance, a researcher may modify the ligand to reduce clashes with receptor residues. This might involve altering nearby groups or adjusting torsion angles.), and so on.

As another example, an optimal conformation may be selected from the conformation pool to guide the researcher to modify the structure of the ligand for achieve the optimal binding affinity when binding with a protein. The optimal conformation may be selected based on (1) a conformation energy of the selected conformation and (2) an amount of energy needed to transition the first molecular conformation to the selected conformation. It means both the resulting energy of the target conformation and the required energy to transition the initial conformation (e.g., in aqueous solution) to the target conformation are considered when selecting the optimal conformation. In some embodiments, the optimal conformation may be the conformation that has the most frequent occurrence of the bond rotation angles from the conformation pool.

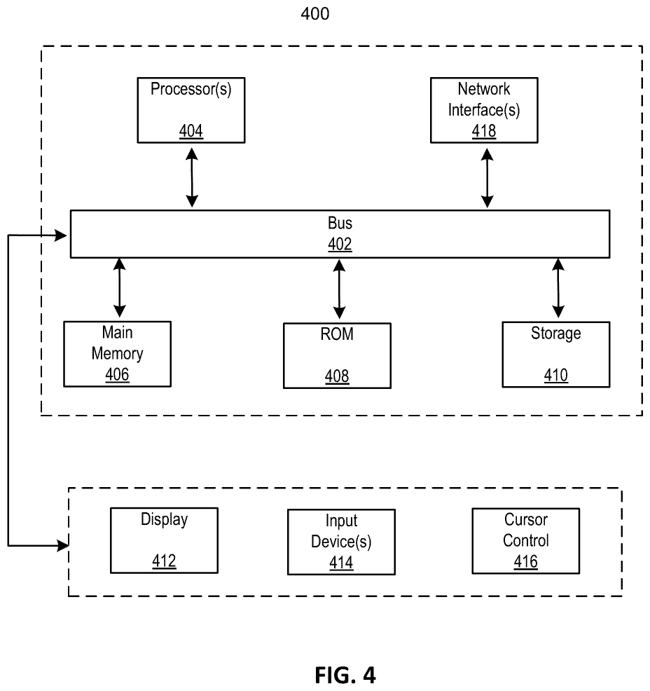

FIG. 4 illustrates a block diagram of an example computer system 400 in which any of the embodiments described herein may be implemented. The computer system 400 includes a bus 402 or other communication mechanisms for communicating information, one or more hardware processors 404 coupled with bus 402 for processing information. Hardware processor(s) 404 may be, for example, one or more general-purpose microprocessors.

The computer system 400 also includes a main memory 406, such as a random access memory (RAM), cache and/or other dynamic storage devices, coupled to bus 402 for storing information and instructions to be executed by processor 404. Main memory 406 also may be used for storing temporary variables or other intermediate information during execution of instructions to be executed by processor 404. Such instructions, when stored in storage media accessible to processor 404, render computer system 400 into a special-purpose machine that is customized to perform the operations specified in the instructions.

The computer system 400 further includes a read-only memory (ROM) 408 or other static storage device coupled to bus 402 for storing static information and instructions for processor 404. A storage device 410, such as a magnetic disk, optical disk, or USB thumb drive (Flash drive), etc., is provided and coupled to bus 402 for storing information and instructions.

The computer system 400 may be coupled via bus 402 to a display 412, such as a cathode ray tube (CRT) or LCD display (or touch screen), for displaying information to a computer user. An input device 414, including alphanumeric and other keys, is coupled to bus 402 for communicating information and command selections to processor 404. Another type of user input device is cursor control 416, such as a mouse, a trackball, or cursor direction keys for communicating direction information and command selections to processor 404 and for controlling cursor movement on display 412. This input device typically has two degrees of freedom in two axes, a first axis (e.g., x) and a second axis (e.g., y), that allows the device to specify positions in a plane. In some embodiments, the same direction information and command selections as cursor control may be implemented via receiving touches on a touch screen without a cursor.

The computing system 400 may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “module,” as used herein, refers to logic embodied in hardware or firmware, or to a collection of software instructions, possibly having entry and exit points, written in a programming language, such as, for example, Java, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules or computing device functionality described herein are preferably implemented as software modules, but may be represented in hardware or firmware. Generally, the modules described herein refer to logical modules that may be combined with other modules or divided into sub-modules despite their physical organization or storage.

The computer system 400 may implement the techniques described herein using customized hard-wired logic, one or more ASICs or FPGAs, firmware and/or program logic which in combination with the computer system causes or programs computer system 400 to be a special-purpose machine. According to one embodiment, the techniques herein are performed by computer system 400 in response to processor(s) 404 executing one or more sequences of one or more instructions contained in main memory 406. Such instructions may be read into main memory 406 from another storage medium, such as storage device 410. Execution of the sequences of instructions contained in main memory 406 causes processor(s) 404 to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, such as storage device 410. Volatile media includes dynamic memory, such as main memory 406. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between non-transitory media. For example, transmission media includes coaxial cables, copper wire and fiber optics, including the wires that comprise bus 402. Transmission media can also take the form of acoustic or light waves, such as those generated during radio-wave and infra-red data communications.

Various forms of media may be involved in carrying one or more sequences of one or more instructions to processor 404 for execution. For example, the instructions may initially be carried on a magnetic disk or solid state drive of a remote computer. The remote computer can load the instructions into its dynamic memory and send the instructions over a telephone line using a modem. A modem local to computer system 400 can receive the data on the telephone line and use an infra-red transmitter to convert the data to an infra-red signal. An infra-red detector can receive the data carried in the infra-red signal and appropriate circuitry can place the data on bus 402. Bus 402 carries the data to main memory 406, from which processor 404 retrieves and executes the instructions. The instructions received by main memory 406 may retrieves and executes the instructions. The instructions received by main memory 406 may optionally be stored on storage device 410 either before or after execution by processor 404.

The computer system 400 also includes a communication interface 418 coupled to bus 402. Communication interface 418 provides a two-way data communication coupling to one or more network links that are connected to one or more local networks. For example, communication interface 418 may be an integrated services digital network (ISDN) card, cable modem, satellite modem, or a modem to provide a data communication connection to a corresponding type of telephone line. As another example, communication interface 418 may be a local area network (LAN) card to provide a data communication connection to a compatible LAN (or WAN component to communicated with a WAN). Wireless links may also be implemented. In any such implementation, communication interface 418 sends and receives electrical, electromagnetic or optical signals that carry digital data streams representing various types of information.

A network link typically provides data communication through one or more networks to other data devices. For example, a network link may provide a connection through local network to a host computer or to data equipment operated by an Internet Service Provider (ISP). The ISP in turn provides data communication services through the world wide packet data communication network now commonly referred to as the “Internet”. Local network and Internet both use electrical, electromagnetic or optical signals that carry digital data streams. The signals through the various networks and the signals on network link and through communication interface 418, which carry the digital data to and from computer system 400, are example forms of transmission media.

The computer system 400 can send messages and receive data, including program code, through the network(s), network link and communication interface 418. In the Internet example, a server might transmit a requested code for an application program through the Internet, the ISP, the local network and the communication interface 418.

The received code may be executed by processor 404 as it is received, and/or stored in storage device 410, or other non-volatile storage for later execution.

Each of the processes, methods, and algorithms described in the preceding sections may be embodied in, and fully or partially automated by, code modules executed by one or more computer systems or computer processors comprising computer hardware. The processes and algorithms may be implemented partially or wholly in application-specific circuitry.

The various features and processes described above may be used independently of one another, or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language, such as, among others, “can,” “could,” “might.” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be removed, executed out of order from that shown or discussed, including substantially concurrently or in reverse order, depending on the functionality involved, as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the subject matter has been described with reference to specific example embodiments, various modifications and changes may be made to these embodiments without departing from the broader scope of embodiments of the present disclosure. Such embodiments of the subject matter may be referred to herein, individually or collectively, by the term “invention” merely for convenience and without intending to voluntarily limit the scope of this application to any single disclosure or concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

It will be appreciated that an “engine,” “system,” “data store,” and/or “database” may comprise software, hardware, firmware, and/or circuitry. In one example, one or more software programs comprising instructions capable of being executable by a processor may perform one or more of the functions of the engines, data stores, databases, or systems described herein. In another example, circuitry may perform the same or similar functions. Alternative embodiments may comprise more, less, or functionally equivalent engines, systems, data stores, or databases, and still be within the scope of present embodiments. For example, the functionality of the various systems, engines, data stores, and/or databases may be combined or divided differently.

“Open source” software is defined herein to be source code that allows distribution as source code as well as compiled form, with a well-publicized and indexed means of obtaining the source, optionally with a license that allows modifications and derived works.

The data stores described herein may be any suitable structure (e.g., an active database, a relational database, a self-referential database, a table, a matrix, an array, a flat file, a documented-oriented storage system, a non-relational No-SQL system, and the like), and may be cloud-based or otherwise.

As used herein, the term “or” may be construed in either an inclusive or exclusive sense. Moreover, plural instances may be provided for resources, operations, or structures described herein as a single instance. Additionally, boundaries between various resources, operations, engines, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

Although the invention has been described in detail for the purpose of illustration based on what is currently considered to be the most practical and preferred implementations, it is to be understood that such detail is solely for that purpose and that the invention is not limited to the disclosed implementations, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment. A component being implemented as another component may be construed as the component being operated in a same or similar manner as the another component, and/or comprising same or similar features, characteristics, and parameters as the another component.

The phrases “at least one of,” “at least one selected from the group of,” or “at least one selected from the group consisting of,” and the like are to be interpreted in the disjunctive (e.g., not to be interpreted as at least one of A and at least one of B).

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment, but may be in some instances. Furthermore, the particular features, structures, or characteristics may be combined in any suitable manner in one or more embodiments.

What is claimed is:

1. A computer-implemented method, comprising:
executing an iterative process using a first molecular conformation of a ligand molecule to obtain a conformation pool, wherein the iterative process comprises:
determining a first conformation energy of the first molecular conformation;
detecting one or more rotatable bonds in the first molecular conformation based on geometry information of the first molecular conformation;
randomly sampling, using a Monte Carlo sampling algorithm, a rotatable bond from the one or more detected rotatable bonds and rotating the rotatable bond to obtain a second molecular conformation;
determining a second conformation energy of the second molecular conformation;
in response to the second conformation energy being less than the first conformation energy, storing the second conformation in a conformation pool;
in response to the second conformation energy being greater than the first conformation energy, determining a probability to store the second conformation in the conformation pool, wherein the probability is exponentially inversely related to a difference between the first conformation energy and the second conformation energy;
replacing the first molecular conformation with the second molecular conformation if the second conformation is stored in the conformation pool; and
continuing the iterative process until an exit condition is met; and
generating binding guidance for the first molecular conformation based on conformations stored in the conformation pool.

2. The computer-implemented method of claim 1, wherein the generating the binding guidance comprises:
determining one or more key rotatable bonds of the first molecular conformation based on bond rotation angle statistics of conformations in the conformation pool, wherein the one or more key rotatable bonds correspond to rotation angles with the least number of appearances in the conformation pool; and
visualizing, on a graphic user interface (GUI), the first molecular conformation by highlighting the one or more key rotatable bonds and displaying corresponding statistics in a histogram.

3. The computer-implemented method of claim 2, wherein the highlighting the one or more key rotatable bonds comprises:
displaying the one or more key rotatable bonds using a color different from a color of other rotatable bonds in the first molecular conformation.

4. The computer-implemented method of claim 1, wherein the generating the binding guidance comprises:
generating conformation entropy based on conformations in the conformation pool.

5. The computer-implemented method of claim 1, further comprising:
converting cartesian coordinates of atoms in the first molecular conformation into internal coordinates before detecting the one or more rotatable bonds in the first molecular conformation.

6. The computer-implemented method of claim 1, wherein the determining the second conformation energy of the second molecular conformation comprises:
inputting the second molecular conformation into a pre-trained deep neural network to evaluate conformation energies of given molecular conformations, wherein the pre-trained deep neural network is trained with labeled training data comprising:
a plurality of molecular structures represented by atomic coordinates, and corresponding quantum mechanical calculated energies as labels.

7. The computer-implemented method of claim 1, wherein the pre-trained deep neural network comprises ANI-2 (ANAKIN-ME model version 2).

8. The computer-implemented method of claim 1, wherein the detecting one or more rotatable bonds in the first molecular conformation comprises:
inputting the geometry information of the first molecular conformation into a software application to check bond types and determine whether a bond is in a ring or has other geometrical constrains.

9. The computer-implemented method of claim 8, wherein the software application comprises AutoDockTools or RDKit.

10. The computer-implemented method of claim 1, wherein the rotating the rotatable bond to obtain the second molecular conformation comprises:
rotating the rotatable bond by a degree using molecular modeling software.

11. The computer-implemented method of claim 1, further comprising:
identifying key rotatable bonds that are most difficult to rotate based on conformations in the conformation pool; and
creating energy barriers for the key rotatable bonds before binding the ligand molecule to a protein molecule or a polymer.

12. The computer-implemented method of claim 1, wherein the generating binding guidance for the first molecular conformation comprises:
selecting one conformation from the conformation pool based on (1) a conformation energy of the selected conformation and (2) an amount of energy needed to transition the first molecular conformation to the selected conformation.

13. The computer-implemented method of claim 1, wherein the continuing the iterative process until the exit condition is met comprises:
tracking an absolute difference between the first conformation energy and the second conformation energy for a plurality of steps in the iterative process; and
exiting the iterative process when variations of the tracked absolute differences remain below a threshold for the plurality of steps.

14. A system comprising:
one or more processors configured to:
execute an iterative process for a first molecular conformation to obtain a conformation pool, wherein the iterative process comprises:
determine a first conformation energy of the first molecular conformation
detect one or more rotatable bonds in the first molecular conformation based on geometry information of the first molecular conformation
randomly sample, using a Monte Carlo sampling algorithm, a rotatable bond from the one or more detected rotatable bonds and rotating the rotatable bond to obtain a second molecular conformation
determine a second conformation energy of the second molecular conformation
in response to the second conformation energy be less than the first conformation energy, storing the second conformation in a conformation pool
in response to the second conformation energy be greater than the first conformation energy, determining a probability to store the second conformation in the conformation pool, wherein the probability is exponentially inversely related to a difference between the first conformation energy and the second conformation energy;
replace the first molecular conformation with the second molecular conformation if the second conformation is stored in the conformation pool; and
continue the iterative process until an exit condition is met; and
generate binding guidance for the first molecular conformation based on conformations stored in the conformation pool.

15. The system of claim 14, wherein the generating the binding guidance comprises:
determining one or more key rotatable bonds of the first molecular conformation based on bond rotation angle statistics of conformations in the conformation pool, wherein the one or more key rotatable bonds correspond to rotation angles with the least number of appearances in the conformation pool; and
visualizing, on a graphic user interface (GUI), the first molecular conformation by highlighting the one or more key rotatable bonds and displaying corresponding statistics in a histogram.

16. The system of claim 15, wherein the highlighting the one or more key rotatable bonds comprises:
displaying the one or more key rotatable bonds using a color different from a color of other bonds in the first molecular conformation.

17. The system of claim 14, wherein the generating the binding guidance comprises:
generating conformation entropy based on conformations in the conformation pool.

18. The system of claim 14, further comprising:
converting cartesian coordinates of atoms in the first molecular conformation into internal coordinates before detecting the one or more rotatable bonds in the first molecular conformation.

19. The system of claim 14, wherein the determining the second conformation energy of the second molecular conformation comprises:
inputting the second molecular conformation into a pre-trained deep neural network to evaluate conformation energies of given molecular conformations, wherein the pre-trained deep neural network is trained with labeled training data comprising:
a plurality of molecular structures represented by atomic coordinates, and corresponding quantum mechanical calculated energies as labels.

20. A non-transitory computer-readable medium storing a set of instructions, the set of instructions comprising:
one or more instructions that, when executed by one or more processors of a device, cause the device to:
execute an iterative process for a first molecular conformation to obtain a conformation pool, wherein the iterative process comprises:
determine a first conformation energy of the first molecular conformation
detect one or more rotatable bonds in the first molecular conformation based on geometry information of the first molecular conformation
randomly sample, using a Monte Carlo sampling algorithm, a rotatable bond from the one or more detected rotatable bonds and rotating the rotatable bond to obtain a second molecular conformation
determine a second conformation energy of the second molecular conformation
in response to the second conformation energy be less than the first conformation energy, storing the second conformation in a conformation pool
in response to the second conformation energy be greater than the first conformation energy, determining a probability to store the second conformation in the conformation pool, wherein the probability is exponentially inversely related to a difference between the first conformation energy and the second conformation energy;
replace the first molecular conformation with the second molecular conformation if the second conformation is stored in the conformation pool; and
continue the iterative process until an exit condition is met; and
generate binding guidance for the first molecular conformation based on conformations stored in the conformation pool.
